(19) United States
(12) Patent Application Publication
(10) Pub. No.: US 2024/0001519 A1
(43) Pub. Date: Jan. 4, 2024

(54) Torque Indicator, Torque Limiter Utilizing Torsional Deflection And Methods Thereof

(71) Applicant: ActivOrtho, Inc., Plymouth, MN (US)

(72) Inventors: Alex Peterson, Maple Grove, MN (US); Michael Brenzel, St. Paul, MN (US); Sam Johnson, Maple Grove, MN (US); Paul J. Hindrichs, Plymouth, MN (US)

(73) Assignee: ActivOrtho, Inc., Plymouth, MN (US)

(21) Appl. No.: 18/251,700

(22) PCT Filed: Nov. 2, 2021
(86) PCT No.: PCT/US2021/072192

§ 371 Date: May 3, 2023

Related U.S. Application Data
(60) Provisional application No. 63/109,246, filed on Nov. 3, 2020.

(51) Int. Cl. B25B 23/142

(52) U.S. Cl. CPC B25B 23/1427; A61B 17/86

(57) ABSTRACT

A torque limiter and/or torque indicator having a distal component, a handle and a torsion component therebetween. The torsion wire can be comprised of a super elastic material that provides a torque plateau preventing over-torquing of an item to be driven, e.g., a bone screw. The torsion component can be a torsion wire or can be a torsion spring. The torsion component can be a single wire or multiple wires. The torsion component can be offset from the axis of rotation of the device.

### RELATED APPLICATIONS

This application claims benefit of and priority to U.S. Provisional Application Ser. No. 63/109,246 filed Nov. 3, 2020 entitled Torque Indicator And Limiter For Compression Screw, which is hereby incorporated herein by reference in its entirety.

### FIELD OF THE INVENTION

The present disclosure relates generally to a driver incorporating a torque indicator and/or torque limiter and more specifically to a torque indicator and/or torque limiter suitable for use in surgical procedures.

### BACKGROUND OF THE INVENTION

It is common for a surgeon or other medical practitioner to insert fasteners into the human body to promote proper healing. For example, orthopedists often treat a variety of different types of bone fractures and other skeletal conditions by installing bone screws, rod and cap screws, or other fasteners in the affected area, to stabilize the bone, to secure implants, or for other therapeutic purposes. Bone screws or other fasteners may require predrilled and tapped holes or may be self-drilling and self-tapping.

When installing a screw or other fastener into bone, it is important that the bone not be subject to excessive torque. Excessive torque may “strip” threads in the bone or may otherwise damage the bone. Further excessive torque may damage screws or other fasteners, for example, by stripping the head of a fastener made from a delicate bioabsorbable material. Thus, applying a limited amount of torque is important in the surgical setting

While existing torque limiters may be acceptable for certain applications, they are poorly suited for use in surgical procedures, such as installing screws or other fasteners into bone. Many existing torque limiters are not resilient to certain sterilization techniques. For example, some existing torque limiters require, or operate best with, lubricating oils that degrade at high temperatures. Thus, such torque limiters are often incompatible with autoclaves, which typically use high-temperature steam to sterilize surgical instruments. Other designs may not be compatible with ethylene oxide (ETO) and/or Gamma sterilization. Many existing torque limiters are also quite complicated. Such complexity may lead to unacceptable size and weight characteristics, increased occurrence of failures and malfunctions, and high costs. Indeed, many existing torque limiters are quite unsuited for single-use applications given their complexity and resultant cost. Finally, many existing torque limiters are not as accurate and precise as desired at the low torque levels commonly used in surgical procedures.

Accordingly, there is a need for an improved torque indicator/limiter suitable for use in surgical procedures that overcomes the shortcomings of prior designs.

### SUMMARY OF THE INVENTION

The present application discloses a device that serves as a torque indicator that allows a user visualize when a given torque amount is achieved.

The present application discloses a device that serves as a torque limiter which limits the amount of torque that can be imparted into a driven item, e.g., a bone screw; has an automatic release of torque at a certain deflection; has an automatic release of torque at a certain force value; and, resets to reapply torque automatically,

The present application discloses torque indicator and/or limiter devices which rely on a mechanism based on minimizing friction between parts. It is based on torsional deflection of, for example, a tube, cylinder, bar or wire. The device exhibits the same force deflection characteristics over multiple uses.

The present application discloses a torque indicator and/or limiter that incorporates super-elastic materials which provide the user with inherent protection against over-torquing a driven item, e.g., a bone screw. Super elastic materials, e.g., Nitinol provide high recoverable strain which can improve the design window of the angular deflection by providing a plateau, which plateau can be set to match the targeted torque limit.

The present application discloses a torque indicator and/or limiter that is sterilizable with steam temperatures up to 150 degrees C., or through the use of ETO, Gama, Ebeam, etc. sterilization.

The present application discloses a torque indicator and/or limiter that is cost effective to manufacture and suitable for disposable use.

A torque limiter and indicator device according to the invention may include a distal component, a handle, a torsion component connecting the distal component and the handle, the torsion component having a predetermined torque profile, the distal component and the handle being rotatable relative to each other subject to resistance from the predetermined torque profile of the torsion component and wherein the predetermined profile has a plateau.

A torque indicator device according to the invention may include a distal component, a handle, a torque component connecting the distal component and the handle, the torque component being offset from a longitudinal axis of the distal component and the handle, the torque component comprised of a wire having a first lever arm member at one end of the wire and a second lever arm member at an opposite end of the wire and the first wire being at an angle relative to an axis of the second lever arm.

A method to limit torque applied to a driven element according to the invention may include providing a device having a distal component connected to a super elastic torsion mechanism, applying torque to the distal component by applying torque to the super elastic torsion mechanism, and continue applying torque to the distal component until torque plateaus according to a torque profile of the super elastic torsion mechanism.

### BRIEF DESCRIPTION OF THE DRAWINGS

These and other aspects, features and advantages of which embodiments of the invention are capable of will be apparent and elucidated from the following description of embodiments of the present invention, reference being made to the accompanying drawings, in which

FIG. 1 is a perspective view of an embodiment of the invention;

FIG. 2 is a perspective view of an embodiment of the invention;

FIG. 3 is cross-sectional view of the embodiment of FIGS. 1 and 2;

FIG. 4 is an assembly view of the embodiment of FIGS. 1 and 2;

FIG. 5 is a view of an embodiment of a torsion wire in accordance with the invention;

FIG. 6 is a chart showing torque versus angular deflection in accordance with an embodiment of the invention;

FIG. 7 is a perspective view of an embodiment of the invention;

FIG. 8 is a perspective view of an embodiment of the invention;

FIG. 9 is a cross-sectional view of an embodiment of the invention;

FIG. 10 is a cross-sectional view taken along lines 10-10 of FIG. 9;

FIG. 11 is a cross-sectional view of an embodiment of the invention;

FIG. 12 is a cross-sectional view of taken along lines 12-12 of FIG. 11;

FIG. 13 is a view of an embodiment of the invention taken along lines 13-13 of FIG. 11;

FIG. 14 is a view of an embodiment of the invention similar to FIG. 13;

FIG. 15 is a perspective view of an embodiment of the invention;

FIG. 16 is a assembly view of an embodiment of the invention;

FIG. 17 is a exposed view of the embodiment of FIGS. 15 and 16;

FIG. 18 is a cross-sectional view of the embodiment of FIGS. 15 and 16;

FIG. 19 is a graph of steel spring behavior in accordance with an embodiment of the invention; and,

FIG. 20 is a graph of shape memory spring behavior in accordance with an embodiment of the invention.

### DETAILED DESCRIPTION

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

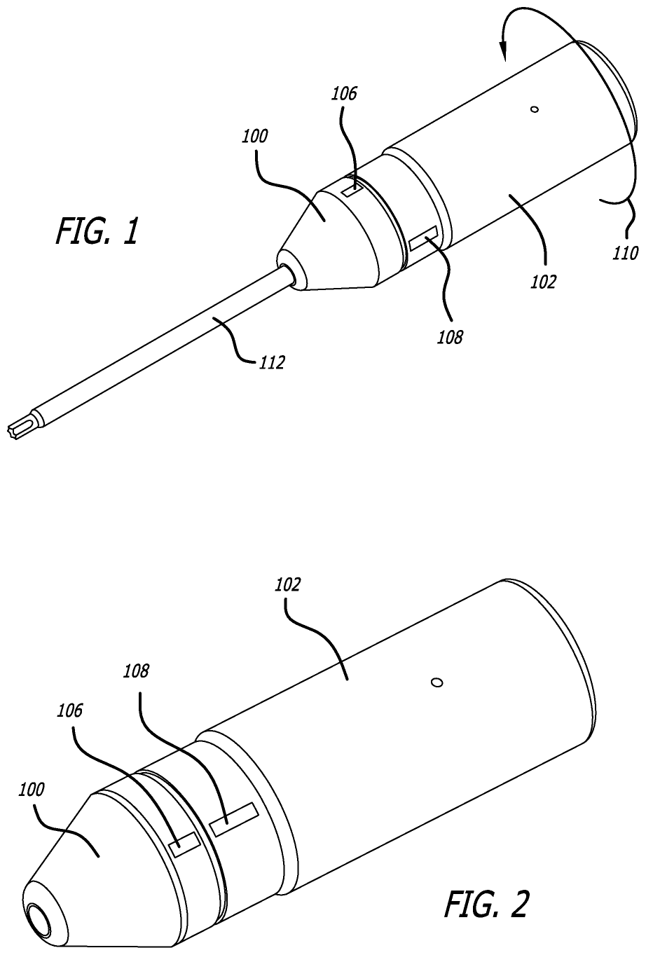

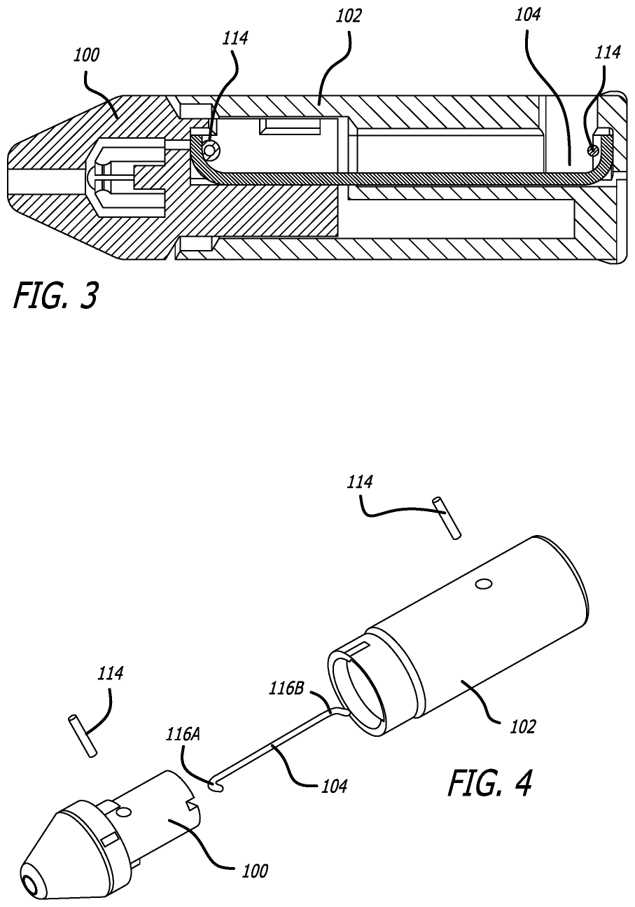

Referring to FIGS. 1-4, one embodiment of torque limiter or torque driver may have three main components, a distal component 100, a torsion wire 104 and a handle 102. When the three components are assembled as shown in FIGS. 3 and 4, the handle 102 is rotatable 110 relative to the distal component 100. The components can be assembled so that the handle is rotatable clockwise or counterclockwise. The distal component is configured to receive a driver 112, which in turn, is configured to mate with a drive head of an item to be driven as is known in the art. In one embodiment, the distal Component 100 has a receptacle which accepts a standard AO driver tip, both retaining the driver tip and preventing its rotation within the distal component 100. One example of an item to be driven is an active compressive bone screw such as described in U.S. Publication No. 2018/0263669 to Peterson et al. entitled Active Compression Apparatus, Method of Assembly and Methods of Use, the contents of which are incorporated herein by reference.

The torsion wire 104 is fashioned with 90-degree bends 116A, 116B at opposite ends of the torsion wire 104. The end of these bends 116A, 1166 engage slotted features in the distal component 100 and handle 102, respectively. In other words, one end of the torsion wire 104 is inserted and secured to the distal component 100 and the opposite end of the torsion wire 104 is inserted and secured to the handle 102. Pins 114 inserted through the body of the distal component 100 and the handle 102 serve to secure the torsion wire 104, both axially and rotationally in each of the distal component 100 and the handle 102 so that turning the handle 102 relative to the distal component will impart torsion on the torsion wire 104. Once assembled, in one embodiment, the only component attaching the handle 102 to the distal component 100 is the torsion wire 104.

The distal component 100 and the handle 102 each have a marker (or indicator) 106, 108, respectively, disposed thereon. When the device is at rest, the markers 106 will be unaligned as shown in FIG. 1. When the handle 102 is rotated 110 and torsion applied to the torsion wire 104, as will occur when driving an item such as a bone screw, the marker 108 on the handle 102 will rotate with the handle 102. When the marker 108 aligns with the marker 106 on the distal component 100, this is an indication that the proper torque is achieved as discussed in more detail below.

In one embodiment rotation 110 is continued till the indication markers 106 and 108 align. In one embodiment a two-finger technique may be used in which the tips of the fingers and thumb of a user rotate the handle 102 approximately 90 degrees to achieve the alignment of the indication markers 106, 108. When the markers 106, 108 are aligned, the torsion wire 104 has been torqued to a predetermined position that exhibits a known force level. In one embodiment there is a stop feature that limits the relative rotation between the distal end and handle component. This stop or limit could correspond to a given deflection amount of the torsion wire 104.

In one embodiment, the torque limiter or torque driver is used to drive an item, e.g., a bone screw, wherein all of the torque applied by the user is transmitted through the torsion wire 104 to the distal component 100 and on to the driver tip, and ultimately, the screw. The torsion wire 104 has elastic properties such that it begins to twist slightly as the applied torque increases. In one embodiment, the range of the applied torque is from 0.1 Nm to 50 Nm and all values in between.

In one embodiment, the amount of deflection of the handle 102 to achieve the desired torque could be set from 1 to 270 degrees of rotation or deflection and all values in between. In one embodiment, it may be desirable to set the amount of rotation or deflection to be 90 degrees or less, which essentially matches the amount a finger and thumb can rotate handle 102 without involving rotation of the wrist. In another embodiment, the rotation or deflection is in the range of 270 degrees to accommodate the rotation of the wrist and fingers.

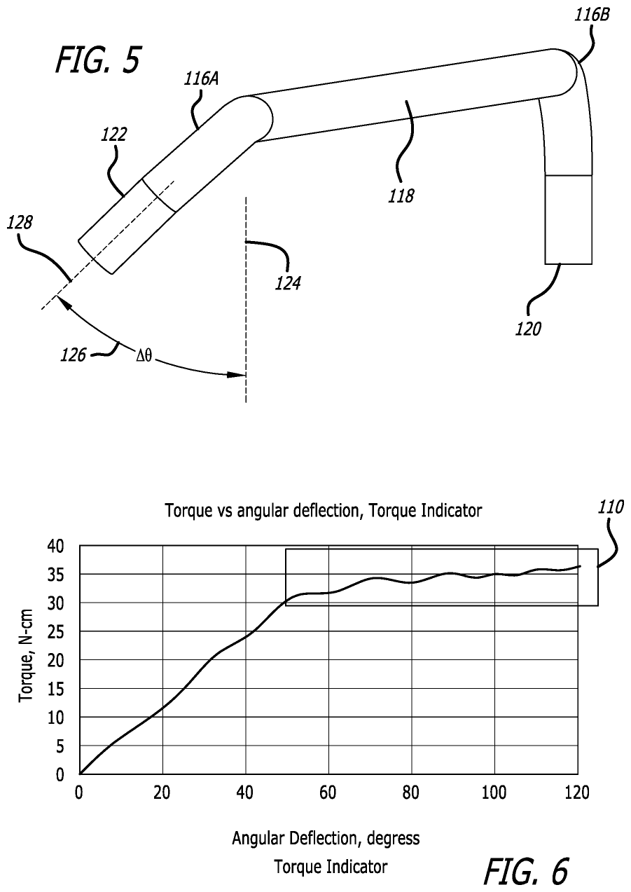

Referring to FIG. 5, the torsion wire 104 includes a torsion section 118 and bent lever ends 120, 122. When the torsion wire 104 is under torsion, the bent lever end 122 is rotated from resting position 124 to an angle of deflection 126. The angle of deflection 126 to leads to bent lever end 122 to be moved to a location 128. When torque is applied, the torque is transmitted to the shaft 118 and is transmitted to the other bent lever end 120.

There is a relationship between the length, diameter, angle of deflection, cross-sectional shape and material properties, etc. of the torque wire 104 that are calculatable to achieve the desired, angular deflection, applied torsion and torsion limit. In other words, the characteristics of the torque wire 104 can be chosen or calculated so that the torque wire 104 provides a desired or predetermined torque profile.

There are multiple materials that could be used for the torsion wire 104. In some embodiments, those materials exhibit the following properties: super elastic, pseudo elastic, elastic and shape memory, and that deform reversibly at high strains. Such materials can include but are not limited to, metal alloys, polymers, rubbers, composites, Nitinol, carbon and others.

In one embodiment the torsion wire 104 can be a hollow tube. In some embodiments, the cross-sectional shape of the torsion wire 104 can be round, ellipse, rectangle, square, triangle, hexagon, or combinations thereof.

In embodiment the torsion wire 104 comprises a super elastic material such as Nitinol.

As known to one of ordinary skill in the art, materials used for springs, such as stainless steel, have a torque profile that is demonstrates linear proportional behavior when comparing the amount of strain or deformation exerted on that material to the amount of the resulting force or torsional resistance, provided the amount of exerted strain or deformation is below the yield strength of the material. However, super elastic materials, such as nitinol, exhibit a different stress vs strain curve or torque profile.

Nitinol behaves essentially identically to typical metals up to about 1% strain. However, between about 1% and 8% strain, the slope of the stress strain curve for Nitinol becomes much less than the initial slope. This property is advantageous in one embodiment of the torque indicator or torque limiter device, in that the sensitivity of rotating the handle 102 to increasing torque is much lower. In other words, when the torsion wire comprises a super-elastic material, e.g., Nitinol, the target torque is approached rapidly as one turns the handle 102 but in the torque range of interest, the torque changes much more slowly with increased turning of the handle 102.

Referring to FIG. 6, in one embodiment, there is a torque vs. deflection curve or torque profile that is substantially linear between zero degrees and about 60 degrees of rotation of the handle 102. However, the torque vs. deflection curve levels of or plateaus upon further rotation of the handle 102 from 60 to 120 degrees. There is only marginal increase in the torque in that range of deflection. This helps prevent inadvertent over-torquing of the item being driven, e.g., a bone screw.

Another embodiment of the torque limiter or torque driver of the present application includes the following features: cannulation, off-center or offset application of torque thus providing a mechanical advantage, multiple numbers of torsion wires, adjustable torque responses; bidirectional functionality or unidirectional functionality.

This embodiment utilizes the same torsional strategy as previous embodiments. Components include of distal member, proximal handle member, and torsional wire elements. The torsional resistance member is off the center axis. More than one torsional member can be used. The torsional element is deflected in a manner that limits the amount of deflection and then releases the stored energy limiting the applied torque.

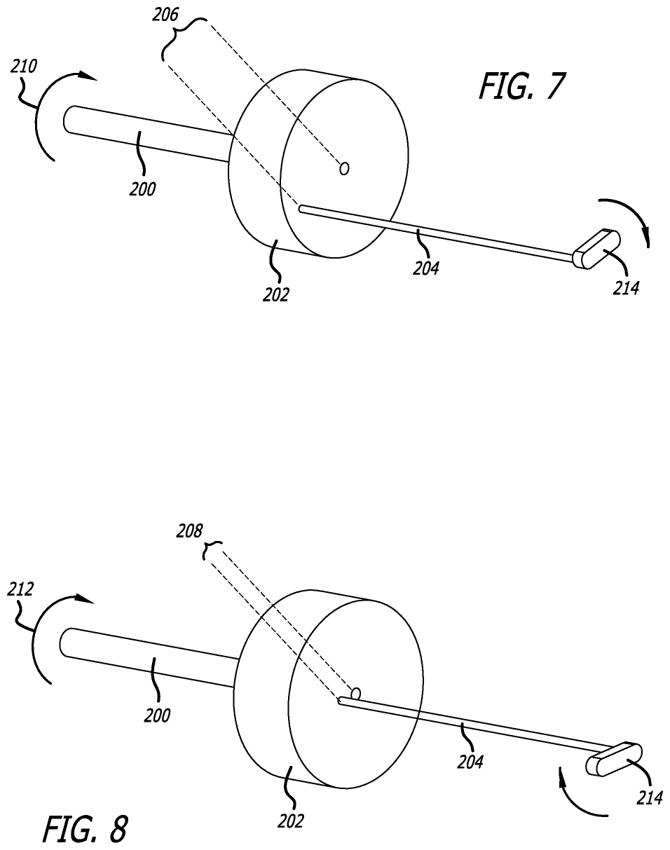

With reference to FIGS. 7 and 8, one embodiment contemplates a torsion wire 204 being offset from a rotational axis of the torque limiter. There is a torque transfer member 200 that extends from a cam 202. The torque transfer member is connected to a distal component. Also connected to the cam 202 is a torsion spring or wire element 204, which, in turn, has a lever arm member 214 (as with previously discussed embodiments). The torsion wire element 204 extends from a handle (via the lever arm member 214) to the cam 202. However, the axis of the torsion wire element 204 is offset from the axis of the torque transfer member 200 by a distance 206 in one embodiment (FIG. 7) and by a distance 208 in another embodiment (FIG. 8). The torque characteristics and the torque 210, 212 exerted by the torque transfer member 200 are different depending on the distance 206, 208 between the center axis of torque transfer member 200 and the center axis of the torsion spring 204. As known to one of skill in the art, these geometries can be adjusted and/or refined to achieve the desired result of input torque and rotation to output torque.

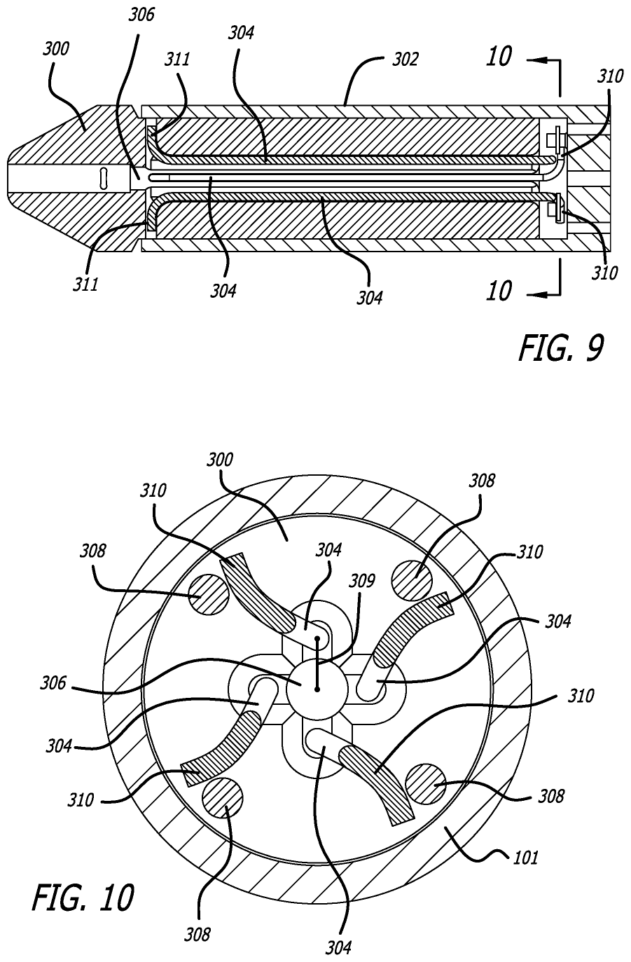

Referring to FIGS. 9 and 10, one embodiment of a torque limiter or torque driver includes a distal component 300 and handle 302 and four torsion wires 304. The torsion wires 304 are placed symmetrically around the circumference of the device. As with previous embodiments, the torsion wires 304 are fixed at their opposite ends via lever arm members 311, 310 to the distal component 300 and handle 302, respectively. In one embodiment, the lever arm members 310 are abutted against pins 308 disposed in the handle 302. In one embodiment the lever arm members 311 are fixed in a compartment of the distal component 300. As with the embodiments of FIGS. 7-8, the torsion wires 304 are offset from the central axis, in this embodiment, by a lever arm distance 309, of the distal component 300 and handle 302, thus enabling torque characteristics as described for the embodiments of FIGS. 7 and 8.

By using multiple torsion wires 304 instead of just one torsion wire, the size (e.g., diameter, shape, etc.) can be reduced for each wire so that collectively the multiple torsion wires 304 produce the torsional characteristics of a single torsion wire design. In addition, since the three torsion wires 304 are offset from the axis of the distal component 300 and handle 302, the device may have a lumen 306 through the center of the device. The lumen 306 allows the device to be cannulated. The lumen 306 enables a user to introduce a guide wire through the device to that the device can be easily moved to a site for inserting a driven member, e.g., a bone screw.

Referring to FIG. 10, when the handle 302 is rotated relative to the distal member 300, torque is applied to each of the torsion wires 304. The torque causes the lever arm members 310 to impinge against the pins 308 and deflect according to the strain and deflection characteristics for each torsion wire 304 as discussed elsewhere in this specification.

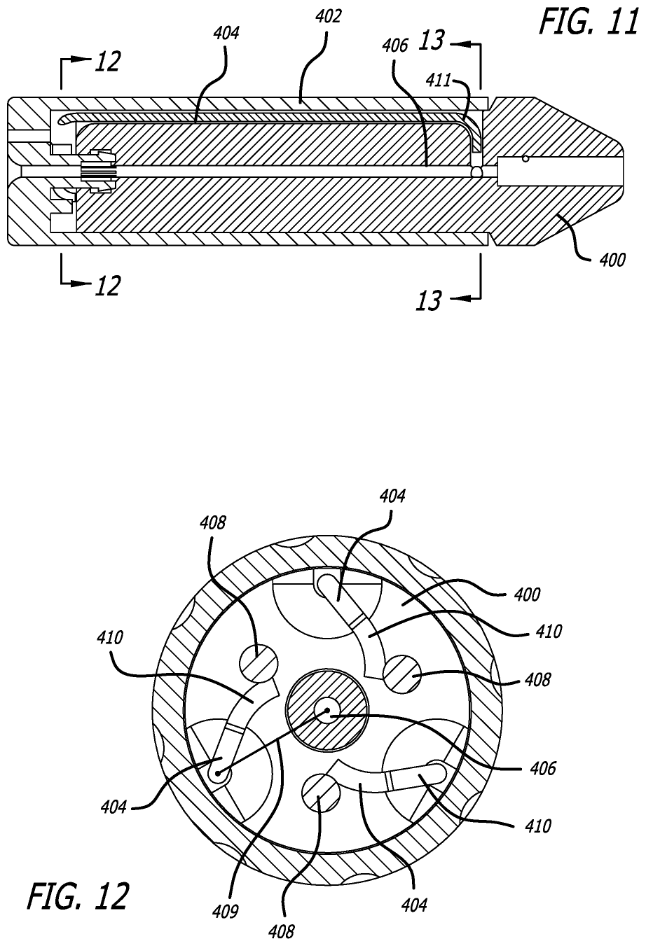

Referring to FIGS. 11 and 12, one embodiment of a torque limiter or torque driver includes a distal component 400 and handle 402 and three torsion wires 304. The torsion wires 404 are placed symmetrically around the circumference of the device. As with previous embodiments, the torsion wires 404 are fixed at their opposite ends via lever arm members 411, 410 to the distal component 400 and handle 302, respectively. In one embodiment, the lever arm members 410 are abutted against pins 408 disposed in the handle 402. In one embodiment the lever arm members 411 are fixed in a compartment of the distal component 400. As with the embodiments of FIGS. 7-8, the torsion wires 404 are offset from the central axis, in this embodiment by a lever arm distance 409, of the distal component 400 and handle 402, thus enabling torque characteristics as described for the embodiments of FIGS. 7 and 8.

By using multiple torsion wires 404 instead of just one torsion wire, the size (e.g., diameter, shape, etc.) can be reduced for each wire so that collectively the multiple torsion wires 404 produce the torsional characteristics of a single torsion wire design. In addition, since the three torsion wires 404 are offset from the axis of the distal component 400 and handle 402, the device may have a lumen 406 through the center of the device. The lumen 406 allows the device to be cannulated. The lumen 406 enables a user to introduce a guide wire through the device to that the device can be easily moved to a site for inserting a driven member, e.g., a bone screw.

Referring to FIG. 12, when the handle 402 is rotated relative to the distal member 400, torque is applied to each of the torsion wires 404. The torque causes the lever arm members 410 to impinge against the pins 408 and deflect according to the strain and deflection characteristics for each torsion wire 404 as discussed elsewhere in this specification.

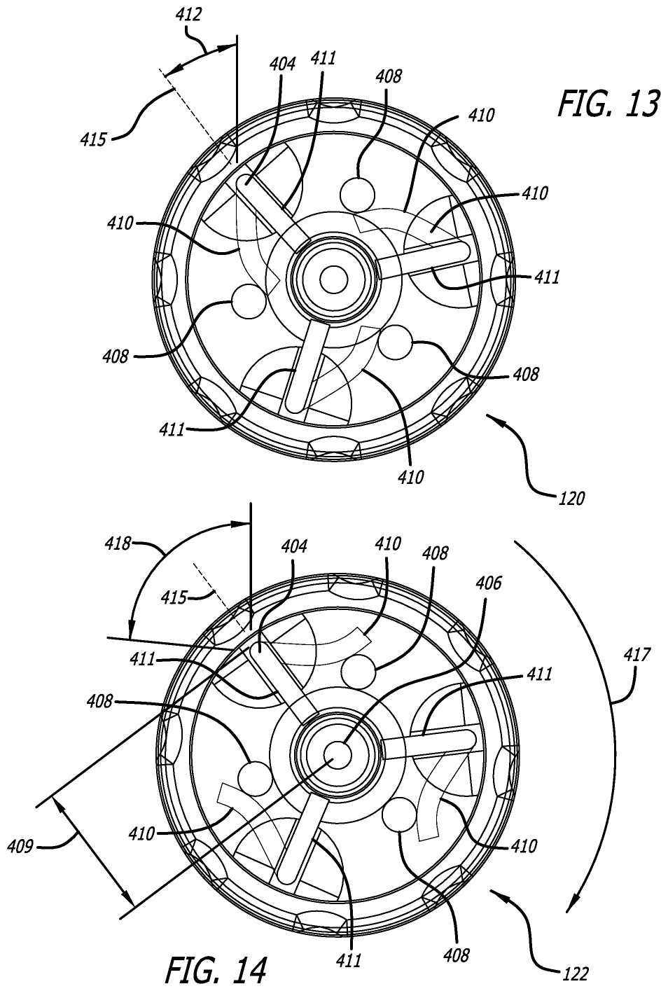

Referring to FIG. 13, the embodiment of FIGS. 11-12 is shown in a resting or unstrained state with an angle 412 existing between the lever arm member 410 and the axis 415 of lever arm member 411 at the opposite end of the torsion wire 404. The angle 412 is predetermined according to the characteristics and desired properties for the torsion wires 404.

Referring to FIG. 14, the embodiment of FIGS. 11-12 is shown in a strained state as a result of the handle 402 being rotated 417 relative to the distal component 400. The torsion wires 404 have been torqued such that the angle of lever arm member 410 relative to the axis 415 of the lever arm member 411 has been changed by an angular delta 418.

The amount of torsion is determined by the previously considered variables in addition to the lever arm distance 409 from the axis of the torsion wire 404 to the axis of rotation (centered in lumen 406).

In this embodiment torque is applied to the driven member, e.g., a bone screw not directly from the rotation of the torsion wires 404, but by applying force from the torsion wires 404 at a levered distance which allows for leverage of the applied force.

In one embodiment, rotation of the handle 402 in a direction opposite to direction 417 causes engagement of the lever arm members 410 in a different way and does not transmit any meaningful torque to the driven member, e.g., the screw.

The lever arm members 410 could be designed to engage the pins 408 such so that they would exert torque in either or both directions of rotation.

In one embodiment, if the rotation of the handle 402 relative to the distal component 400 exceeds a predetermined angle, the lever arm members 410 will move beyond the pins 408 and shall spring back to a non-strained state as shown in FIG. 13.

In one embodiment the torque required to move the arm members 410 beyond the pins 408 is determined by the equation T=N×(F×R), wherein F is the force required to turn the handle 402, N is the number of torsion wires, and R is the lever arm distance as discussed above.

It is often desired to know the value of torque being applied to a driven member. For example, when a bone screw is used to bring fractured bone segments into intimate contact, it is desirable for a user to understand the torque being applied to the screw.

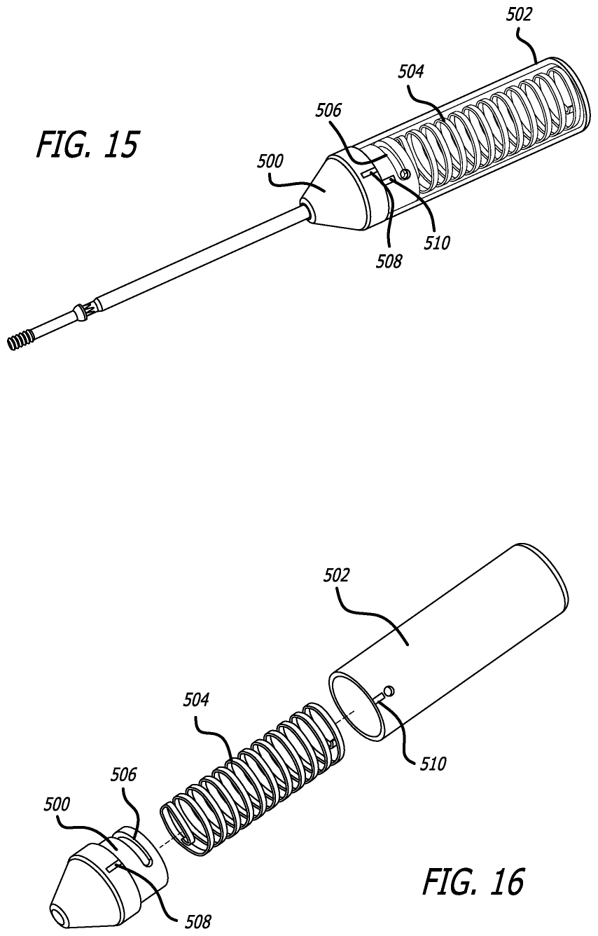

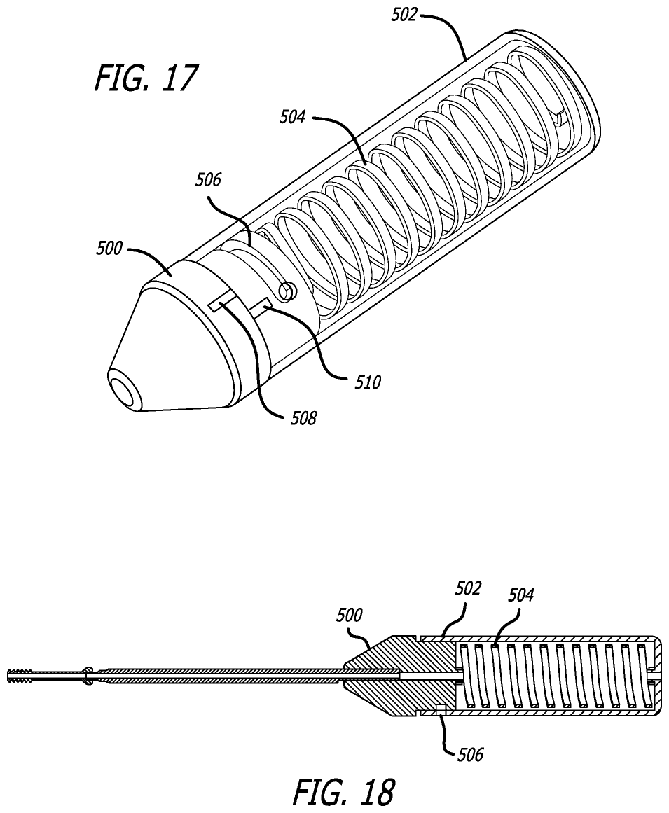

Referring to FIGS. 15-18, one embodiment of a torque indicator device is shown. It has a handle 502, a distal component 500 and a torsional spring 504. I operation, the torque indicator is grasped with the hand and used to drive the screw in a manner similar to previous described embodiments.

During assembly of the device, the torsional spring 504 is pre-loaded, or wound, to a predetermined torque value T. The device is then permanently fastened together, using a pin or other fastening method as known to those of skill in the art to maintain the torsional spring 504 in the preloaded state.

For example, a slot 506 is incorporated into the distal component 500 to receive one end of the torsional spring 504. The opposite end of the torsional spring is secured or fixed to the handle 502. The slot 506 enables the torsional spring 504 to remain in its pre-loaded state upon assembly and in its resting state. However, upon rotation of the handle 502 with a torque that exceeds the predetermined torque value T, the handle 502 will begin to rotate relative to the distal component 500.

In one embodiment, there are markers 508, 510 on the distal component 500 and the handle 502, respectively. When the device is assembled and the torsional spring 504 is in its pre-loaded state, the markers are not aligned, i.e., they are rotationally offset by an angle relative to each other as shown in FIG. 15.

In one embodiment, the rotation limit maintaining the torsional spring 504 at its predetermined torque value T is reflected by the markers 508, 510 being separated by an angle of 45 degrees.

As the user turns the handle to drive the driven member, e.g., driving a bone screw into bone fragments, there is an increasing torque inherent to the tightening of the bone screw. The markers 508, 510 will not move relative to each other until the predetermined torque T exceeded. When the pre-loaded torque T is achieved and exceeded, the markers 508, 510 shall move closer into alignment with each other. This movement communicates to the user that the specified torque has been achieved.

In one embodiment, the marks 508, 510 are aligned, not misaligned. As such when the user tightens the screw beyond the predetermined torque T the markers 508, 510 shall move away from each other not towards each other.

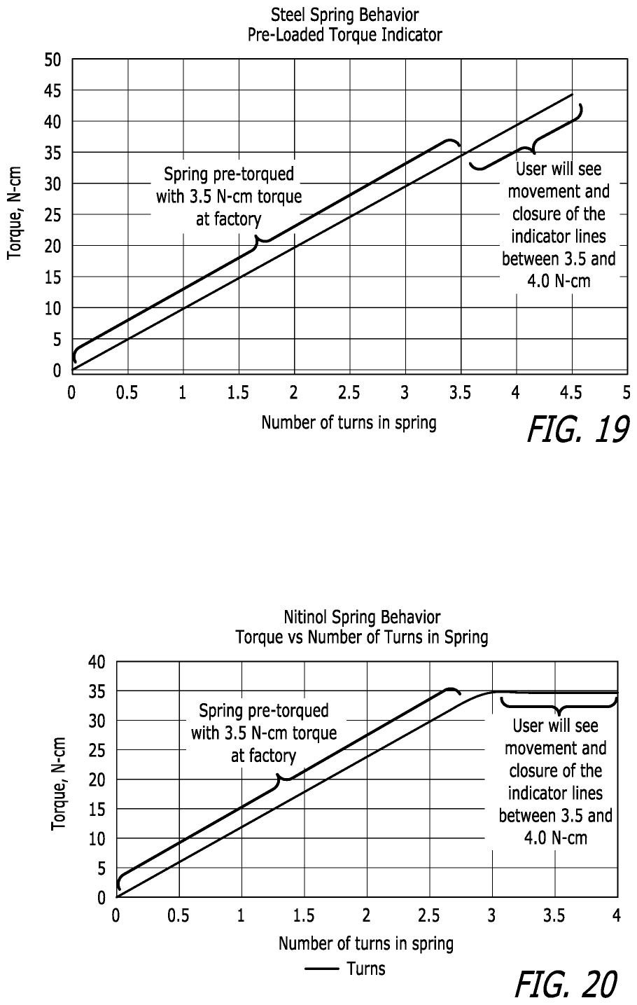

In one embodiment the torsional spring 504 is a steel coil spring and the preload torque T is 3.5N-cm as set forth in FIG. 19. In one embodiment, this means the steel coil spring is pre-wound at a manufacturing site about 3.5 turns.

A preloaded torque T in a steel coil spring provides a characteristic to the device or a torque profile that is similar to the behavior of a torque profile that has a plateau function as described above with respect to torsion wires comprised of super-elastic materials, e.g., Nitinol. No movement of the markers 508, 510 occurs so long as the torque does not exceed the preloaded torque T and, as such, the device operates at a plateau of torque until the torque value T is exceed.

In one embodiment, the torsional spring 504 is comprised of Nitinol as described in FIG. 20. In such an embodiment the plateau characteristic unique to super elastic materials is added. As a result, environmental conditions such as temperature, humidity, etc. that otherwise affect the torsional properties of, for example, a torsional spring made of steel are avoided. This ensures proper and consistent application of preferred torque values to the driven item in essentially all ambient environments.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

1. A torque limiter device comprising:
a distal component;
a handle
a torsion component connecting the distal component and the handle;
the torsion component having a predetermined torque profile;
the distal component and the handle being rotatable relative to each other subject to resistance from the predetermined torque profile of the torsion component;
wherein the predetermined torque profile has a plateau.

2. The torque limiter device according to claim 1, wherein the torsion component is comprised of at least one wire.

3. The torque limiter device according to claim 2, wherein torsion component is at least two wires.

4. The torque limiter device according to claim 1, wherein the torsion component is a torsional spring.

5. The torque limiter according to claim 1, wherein the torsion component is comprised of lever arm members disposed at opposing ends of the torsion component.

6. The torque limiter according to claim 2, wherein one lever arm member is set at an angle relative to an axis of the opposite lever arm member.

7. The torque limiter according to claim 1, wherein the torsion component is offset from an axis of rotation of the distal component and the handle.

8. The torque limiter according to claim 1, wherein a marker is disposed on the handle and the distal component.

9. The torque limiter according to claim 1, further comprising a lumen extending through the distal component and the handle.

10. A torque indicator device comprising:
a distal component;
a handle;
a torque component connecting the distal component and the handle;
the torque component being offset from a longitudinal axis of the distal component and the handle;
the torque component comprised of a wire having a first lever arm member at one end of the wire and a second lever arm member at an opposite end of the wire;
the first wire being at an angle relative to an axis of the second lever arm.

11. A torque indicator device according to claim 10, wherein the torque component is comprised of multiple wires.

12. A torque indicator device according to claim 10, wherein the torque component is comprised of Nitinol.

13. A torque indicator device according to claim 10, wherein the torque component has a torque profile with a plateau limiting torque that is applied to the distal component.

14. A torque indicator device according to claim 10, wherein the second lever arm is situated in the handle to be movable relative to an axis of the first lever arm.

15. A torque indicator device according to claim 14, wherein the first lever arm is secured in the distal component against movement relative to the first lever arm.

16. A method to limit torque applied to a driven element comprising:
providing a device having a distal component connected to a super elastic torsion mechanism;
applying torque to the distal component by applying torque to the super elastic torsion mechanism;
continue applying torque to the distal component until torque plateaus according to a torque profile of the super elastic torsion mechanism.

17. The method according to claim 16, wherein applying the torque comprises applying torque through multiple torsion wires.

18. The method according to claim 16, wherein applying the torque comprises applying torque on an access offset from an axis rotation of the distal component.

19. A torque indicator device comprising:
a distal component;
a handle component;
a torsion component disposed between the distal component and the handle component;
the torsion component having a preloaded torque.

20. A torque indicator device according to claim 19, wherein the torsion component is a coiled spring.
